(19) United States
(12) Patent Application Publication
(10) Pub. No.: US 2013/0103011 A1
(43) Pub. Date: Apr. 25, 2013

(54) System and Method for Lowering IOP by Creation of Microchannels in Trabecular Meshwork Using a Femtosecond Laser

(71) Applicants: Robert Edward Grant, Laguna Beach, CA (US); David Haydn Mordaunt, Los Gatos, CA (US)

(72) Inventors: Robert Edward Grant, Laguna Beach, CA (US); David Haydn Mordaunt, Los Gatos, CA (US)

(21) Appl. No.: 13/405,087

(22) Filed: Feb. 24, 2012

Related U.S. Application Data
(60) Provisional application No. 61/550,286, filed on Oct. 21, 2011.

(51) Int. Cl. A61F 9/008

(52) U.S. Cl. 606/4

(57) ABSTRACT

A system and its method for creating a microchannel in the trabecular meshwork of an eye include a laser unit for generating a laser beam, and an imaging unit for creating an image of the trabecular meshwork. The system also includes a computer which defines the microchannel. A comparator that is connected with the computer then controls the laser unit to move the focal point of the laser beam. This focal point movement is accomplished to create the microchannel, while minimizing deviations of the focal point from the defined microchannel.

This application claims the benefit of U.S. Provisional Patent Application Ser. No. 61/550,286, filed Oct. 21, 2011.

### FIELD OF THE INVENTION

The present invention pertains generally to systems and methods for performing ophthalmic surgical procedures using laser devices. More particularly, the present invention pertains to systems and methods for surgically relieving Intraocular Pressure (IOP) to prevent glaucoma. The present invention is particularly, but not exclusively, useful as a system and method for creating microchannels through the trabecular meshwork or through the iris to thereby avoid using mechanical “shunts” and/or “stents” in the eye for the treatment of glaucoma.

### BACKGROUND OF THE INVENTION

Schlemm's canal is a circular channel in the eye that is located in corneal/scleral tissue at the juncture between these two tissues. Schlemm's canal surrounds the cornea, and its functionality is essentially two-fold. For one, it collects aqueous humor from the anterior chamber of the eye. For another, it takes the aqueous humor that is collected from the anterior chamber and delivers it through anterior ciliary veins to the bloodstream. The net effect of this transfer of aqueous humor from the eye to the bloodstream is to control the intraocular pressure (IOP) inside the eye.

Located between Schlemm's canal and the anterior chamber of the eye is the trabecular meshwork. From a fluid flow perspective, the trabecular meshwork is intended to control the outflow of aqueous humor from the anterior chamber. It happens, however, that resistance to this outflow can be substantially aggravated (i.e. increased) for various reasons. When this happens, an unwanted consequence is an increase in IOP, and the possibility of an onset of glaucoma.

A complication that can contribute to an increase in IOP and glaucoma occurs when a fluid flow restriction develops between the iris and the crystalline lens. Typically, this occurs when the gap between the iris and lens closes or is otherwise obstructed. This may be further aggravated by a narrowing of the region around the trabecular meshwork.

Heretofore, one of the standard treatments for glaucoma has been the placement (implantation) of mechanical shunts or stents into the trabecular meshwork. Specifically, this has been done for the purpose of using the shunts or stents as fluid flow conduits for relieving IOP. These mechanical devices, however, are perceived by the body as foreign objects. And the body responds accordingly. One short term effect of the body's response is that the IOP is actually reduced somewhat. It is, however, only a short term effect. In the longer term, these mechanical devices have been generally ineffective for their intended purpose.

In light of the above, it is an object of the present invention to provide a system and method for creating microchannels in the trabecular meshwork between the anterior chamber and Schlemm's canal that will improve the outflow of aqueous humor from the anterior chamber, to thereby prevent the onset of glaucoma. Another object of the present invention is to create microchannels through the iris to improve fluid flow from the posterior chamber into the anterior chamber to help prevent the onset of glaucoma. Yet another object of the present invention is to provide a system and method for creating microchannels in the anterior portion of an eye to prevent the onset of glaucoma, wherein the microchannels are created by Laser Induced Optical Breakdown (LIOB) to thereby avoid the implantation of foreign mechanical objects in the eye. Still another object of the present invention is to provide a system and method for creating microchannels in an eye that are easy to use, are simple to implement and are comparatively cost effective.

### SUMMARY OF THE INVENTION

In accordance with the present invention, a system and method are provided for creating a microchannel through the trabecular meshwork or the iris of an eye to interconnect the anterior chamber and the posterior chamber of the eye in fluid communication with Schlemm's canal. The purpose of the microchannel(s) is to enhance the functionality of Schlemm's canal by improving the outflow of aqueous fluid from the eye. A consequence of this is the lowering of intraocular pressure (IOP) in the eye. In operation, each microchannel is created by using a laser to perform LIOB in the target tissue (i.e. the trabecular meshwork and/or the iris). Thus, for the present invention LIOB is employed rather than having to introduce mechanical objects into the eye for the intended purpose. It is an important aspect of the present invention that the microchannel(s) are created by a computer controlled laser, wherein the control reference is provided by Optical Coherence Tomography (OCT).

Structurally, the system includes a laser unit for generating and directing a pulsed, femtosecond laser beam along a laser beam path to a focal point. The system also includes an imaging unit that is used for creating a three dimensional image of the trabecular meshwork. Preferably, as indicated above, this imaging unit is an Optical Coherence Tomography (OCT) device of a type that is well known in the pertinent art for the intended purpose. Further, the system includes a computer that is interconnected to both the laser unit and the imaging unit.

As intended for the present invention, a computer program defines the microchannel(s). Specifically, this computer program includes information about the location and dimensions of the intended microchannel(s). Further, the computer program establishes where each microchannel will be located and positioned to interconnect the posterior and anterior chambers of the eye in fluid communication with Schlemm's canal. In detail, a microchannel may be a complete channel that passes through either the trabecular meshwork or the iris. In the specific case of the trabecular meshwork, the microchannel may alternatively be a partial channel that extends only part way into the trabecular meshwork. Also, in the case of the trabecular meshwork, instead of a microchannel, the laser may be used to only heat the tissue for stimulating fluid flow. In the event, any combination of the above stated possibilities can be employed.

During the creation of a microchannel, a comparator that is connected to the imaging unit and to the computer uses information from the computer program to determine whether there is an actual operational deviation of the focal point from the defined microchannel(s). If so, an error signal that is indicative of the deviation is generated, and the focal point of the laser beam is moved to minimize the error signal. In this manner, the focal point of the laser beam is guided by the computer program to alter target tissue in the anterior portion of the eye by Laser Induced Optical Breakdown (LIOB), to thereby create the microchannel(s).

In one embodiment of the present invention, a gonioscope can be used for guiding the laser beam. If used, the gonioscope will include a contact lens that is structurally connected to the laser unit. Also, there will be a deflecting mirror that is mounted on the contact lens for directing the laser beam path toward the trabecular meshwork. Alternatively, it is also envisioned that the laser beam path can be directed to the trabecular meshwork directly through the sclera of the eye.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of a system in accordance with the present invention shown in its operational relationship with an eye, which is shown in cross section;

FIG. 2 is an enlarged view of the anterior chamber angle of an eye, as shown surrounded by the line 2-2 in FIG. 1;

FIG. 3A is a representation of a complete microchannel shown passing through the trabecular meshwork in accordance with the present invention;

FIG. 3B is a representation of a partial microchannel shown passing part way through the trabecular meshwork in accordance with the present invention;

FIG. 4 is a functional block diagram of a closed-loop control system incorporating components of the present invention;

FIG. 5A is a cross-sectional view of an eye (as seen in FIG. 1) showing a laser beam passing through the sclera of an eye and into the trabecular meshwork of the eye for creation of a microchannel; and

FIG. 5B is a cross-sectional view of an eye (as shown in FIG. 5A) showing a laser beam being directed by a gonioscope into the trabecular meshwork of the eye for creation of a microchannel.

### DESCRIPTION OF THE PREFERRED EMBODIMENTS

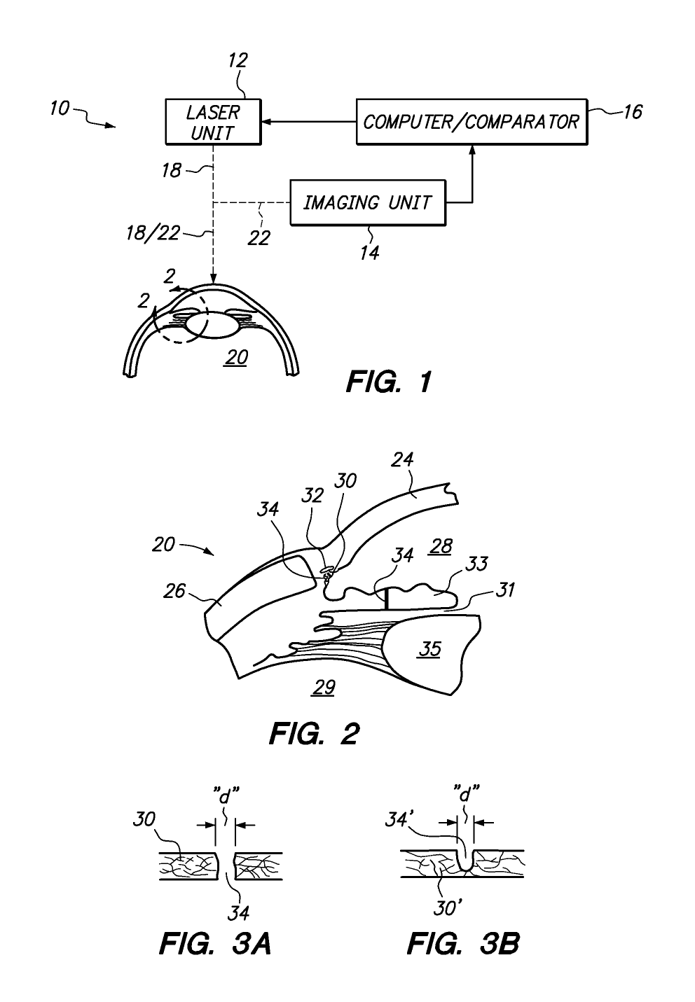

Referring initially to FIG. 1, a system for creating microchannels in the anterior portion of an eye is shown and is generally designated 10. As shown, the system 10 includes a laser unit 12, an imaging unit 14 and a computer/comparator 16. In the system 10, the imaging unit 14 is operationally connected to the computer/comparator 16, and the computer/comparator 16 is connected directly to the laser unit 12. With this combination, the system 10 is used to generate and direct a laser beam 18 toward an eye 20 for an ophthalmic surgical procedure as envisioned for the present invention.

For the purposes of the present invention, the laser unit 12 is capable of generating a so-called “femtosecond” laser beam 18. Thus, the generated laser beam 18 includes a sequence of laser pulses having a very ultra-short duration (e.g. less than approximately 500 fs). Importantly, the laser beam 18 must be capable of performing Laser Induced Optical Breakdown (LIOB) on selected target tissue inside the eye 20. Further, it is important for there to be a precise performance of this LIOB. Such precision requires there be a capability of imaging the target tissue that is to be altered by LIOB.

The imaging unit 14 is preferably a type of device that operates using Optical Coherence Tomography (OCT) techniques. Thus, the imaging unit 14 will include a light source to generate an imaging beam 22 and optics to direct the imaging beam 22 toward the eye 20. In this case, the imaging beam 22 is used to create three dimensional images of selected tissues within the eye 20. As indicated in FIG. 1, these images are then passed to the computer/comparator 16 for use by the computer/comparator 16 in controlling the laser unit 12. As envisioned for the present invention, the precision required for this control will be best appreciated with reference to FIG. 2.

In FIG. 2, the anterior chamber angle and its adjacent anatomical features of the eye 20 are shown. In particular, FIG. 2 identifies the cornea 24 and the sclera 26 of the eye 20. It also identifies the anterior chamber 28 of the eye 20 and the trabecular meshwork 30 that interconnects the anterior chamber 28 with Schlemm's canal 32. In a normal eye 20, aqueous from the anterior chamber 28 passes through the trabecular meshwork 30 and into Schlemm's canal 32. From there, the aqueous is returned to the bloodstream. For various reasons, however, this process may be impeded by tissue structures in the eye 20. If this happens, as envisioned for the system 10 of the present invention, a microchannel 34, or a plurality of microchannels 34, can be created through target tissue in the anterior portion of eye 20 to alleviate this condition (e.g. the onset of glaucoma).

Several situations are of particular interest for the present invention. For one, there is interest in establishing a fluid flow capability from the anterior chamber 28, out through the trabecular meshwork 30 and into Schlemm's canal 32. In this case a laser trabeculoplasty procedure is envisioned wherein microchannels 34 are established into or through the trabecular meshwork 30. Also, a fluid flow capability from the posterior chamber 29 and into the anterior chamber 28 is of interest. Specifically, this additional concern arises when the gap 31 between the iris 33 and the crystalline lens 35, which normally allows for fluid flow, is closed or otherwise becomes occluded. In this case, microchannels 34 may need to be created through the iris 33 in an iridotomy procedure. If required, this will be done to establish fluid flow through the iris 33 from the posterior chamber 29 into the anterior chamber 28.

For purposes of the present invention, microchannels 34 can be employed for either a laser trabeculoplasty procedure or for an iridotomy procedure. With regard to a laser trabeculoplasty procedure, a microchannel 34 can be configured as either a complete microchannel 34 (see FIG. 3A), which passes completely through the trabecular meshwork 30; or as a partial microchannel 34′ (see FIG. 3B) which passes only part way through the trabecular meshwork 30. In either case, the diameter “d” of the microchannel 34 or 34′ will be somewhere in a range between about one hundred microns and approximately four hundred microns (“d”=100 μm→400 μm). Also, the sidewalls of the microchannel 34, 34′, are typically to be burnt in order to prevent closure. Additionally, instead of creating either a complete or a partial microchannel 34, the trabecular meshwork 30 can be heated to stimulate fluid flow through the trabecular meshwork 30. On the other hand, with regard to an iridotomy procedure, a complete microchannel 34 is typically required through the iris 33.

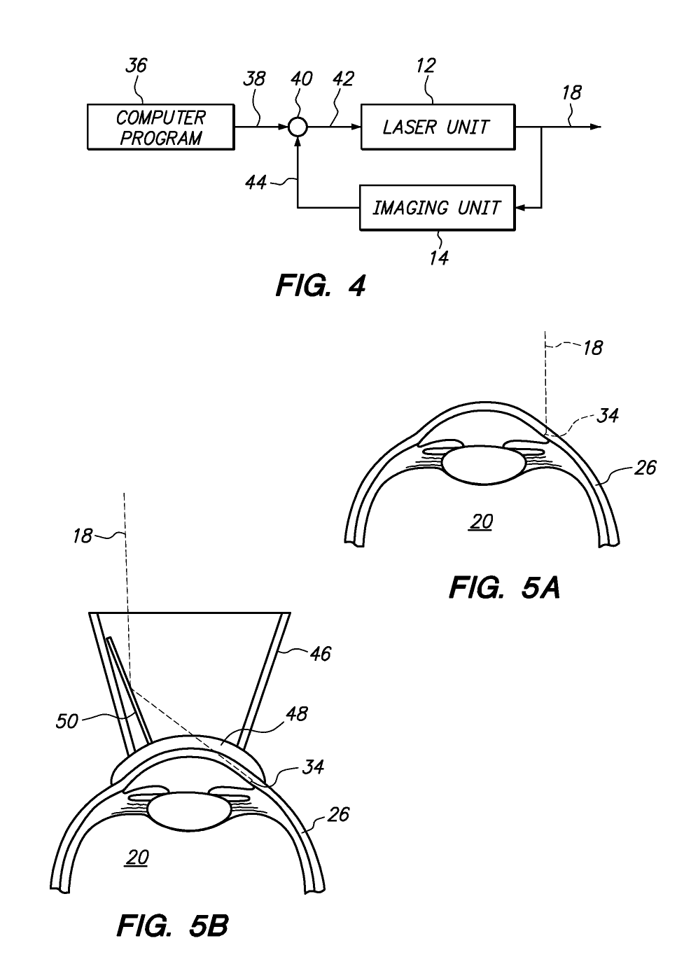

FIG. 4 indicates that the system 10 is intended to be computer-controlled and operated with closed loop feedback. For this operation, a computer program 36 is provided for use with the computer/comparator 16. Specifically, the computer program 36 will include a definition for each of the microchannel(s) 34 that are to be created in the trabecular meshwork 30. This definition will necessarily include the location and the dimensions of each microchannel 34. As envisioned for the present invention, there may be a need for a plurality of such microchannels 34. For example, looking down onto a plan view of the eye 20, it may be desirable to create individual microchannels 34 at, for example, the 2, 4, 8 and 10 o'clock positions. For both procedures (laser trabeculoplasty and iridotomy), the microchannel 34 (34′) can extend through arcs of 180° to 360°. In any case, in order to establish a location for the microchannel 34, as well as for other laser functions, the computer program 36 provides a reference input 38 for the system 10.

In the operation of system 10, the reference input 38 from the computer/comparator 16 (i.e. computer program 36) is sent to a summing point 40. It is then sent from the summing point 40 as an actuating signal 42 for the laser unit 12. Thus, the laser beam 18 is generated as an output from the laser unit 12 in accordance with the actuating signal 42. For guidance and control purposes, the output of the laser unit 12 (i.e. laser beam 18) is monitored by the imaging unit 14. Further, the imaging unit 14 creates three dimensional images that show the effects of LIOB in the trabecular meshwork 30. These images are then used as the basis for generating feedback (error) signals 44 that are returned to the summing point 40. At the summing point 40, the reference input 38 (i.e. definition of microchannel 34) is compared with the feedback (error) signal 44 (i.e. images from the trabecular meshwork 30). This comparison is then used to appropriately adjust the actuating signal 42. As with any closed loop feedback control system, the objective here is to maintain the feedback (error) signal 44 at a null.

Different methods for employing the system 10 are shown in FIG. 5A and FIG. 5B. In FIG. 5A, it is shown that the laser beam 18 can be sent directly through the sclera 26 for LIOB in the trabecular meshwork 30, while the eye 20 is being stabilized. In this case, it may be necessary to hydrate the sclera 26 with topical ointments or injections in a manner that will make the sclera 26 effectively transparent during a procedure. On the other hand, as shown-in FIG. 5B, a gonioscope 46 may be used. As shown, in this alternative method for using system 10, the gonioscope 46 will include a contact lens 48 which can be connected with the laser unit 12 to stabilize the eye 20. It will also include a deflecting mirror 50 that will direct the laser beam 18 onto the trabecular meshwork 30 where the microchannel 34 is to be created by LIOB.

While the particular System and Method for Lowering IOP by Creation of Microchannels in Trabecular Meshwork Using a Femtosecond Laser as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A system for improving the outflow of aqueous fluid from the anterior chamber of the eye to lower the intraocular pressure (IOP) of the eye, the system comprising:
a laser unit for generating a laser beam, and for directing the laser beam along a laser beam path to a focal point in a target tissue to alter the target tissue by Laser Induced Optical Breakdown (LIOB) and establish a fluid flow channel in the target tissue;
an imaging unit for creating an image of the target tissue;
a computer connected to the laser unit, and to the imaging unit, for guiding the focal point of the laser beam in accordance with a predetermined computer program, wherein the computer program defines the fluid flow channel; and
a comparator connected to the imaging unit and to the computer for determining a deviation of the focal point from the defined fluid flow channel to produce an error signal indicative of the deviation, and for moving the focal point to minimize the error signal during creation of the fluid flow channel.

2. A system as recited in claim 1 wherein the laser beam is a pulsed femtosecond laser beam, and the imaging unit is operated using Optical Coherence Tomography (OCT) techniques.

3. A system as recited in claim 1 wherein the fluid flow channel is a microchannel extending through the trabecular meshwork, and wherein the microchannel is positioned to interconnect the anterior chamber of the eye in fluid communication with Schlemm's canal.

4. A system as recited in claim 1 wherein the fluid flow channel is a microchannel extending through the iris of the eye, and wherein the microchannel is positioned to interconnect the posterior chamber of the eye in fluid communication with the anterior chamber of the eye.

5. A system as recited in claim 1 further comprising:
a contact lens connected to the laser unit; and
a deflecting mirror mounted on the contact lens for directing the laser beam path to the target tissue.

6. A system as recited in claim 4 wherein the laser beam path is directed to the target tissue through the sclera of the eye.

7. A closed loop feedback control system for creating a microchannel in a target tissue of an eye which comprises:
a computer with a computer program, wherein the computer program defines the microchannel in the trabecular meshwork for use as a reference input for the system;
a laser unit for generating a laser beam, wherein the laser unit is responsive to an actuating signal from the computer to establish an output for directing the laser beam from the laser unit to a focal point in the target tissue;
an imaging unit for creating an image of the target tissue; and
a comparator for receiving the output from the laser unit, and for receiving the image from the imaging unit, to generate a feedback error signal based on the reference input, wherein the feedback error signal is a measure of a deviation of the focal point of the laser unit from the image of the microchannel created by the imaging unit, and wherein the feedback error signal is used for modifying the actuating signal to the laser unit, with an altered reference input from the computer, to minimize the feedback error signal.

8. A system as recited in claim 7 wherein the microchannel is established to enhance the functionality of Schlemm's canal by improving the outflow of aqueous fluid from the anterior chamber of the eye in order to lower the intraocular pressure (IOP) of the eye.

9. A system as recited in claim 7 wherein the imaging unit is an Optical Coherence Tomography (OCT) device.

10. A system as recited in claim 7 wherein the laser beam is a pulsed femtosecond laser beam.

11. A system as recited in claim 7 wherein the microchannel extends through the trabecular meshwork, and wherein the microchannel is positioned to interconnect the anterior chamber of the eye in fluid communication with Schlemm's canal.

12. A system as recited in claim 7 wherein the microchannel extends through the iris of the eye, and wherein the microchannel is positioned to interconnect the posterior chamber of the eye in fluid communication with the anterior chamber of the eye.

13. A system as recited in claim 7 wherein the laser beam is directed along a laser beam path, and the system further comprises a gonioscope for altering the laser beam path.

14. A method for creating a microchannel in a target tissue in an eye which comprises the steps of:
generating a laser beam;
directing the laser beam along a laser beam path to a focal point in the target tissue;
guiding the focal point of the laser beam in accordance with a predetermined computer program to alter tissue by Laser Induced Optical Breakdown (LIOB) for creation of the microchannel, wherein the computer program defines the microchannel;
creating an image of the target tissue;
locating a placement for the microchannel in the image;
determining a deviation of the focal point from the image of the microchannel in the target tissue;
producing an error signal indicative of the deviation; and
moving the focal point of the laser beam to minimize the error signal during creation of the microchannel.

15. A method as recited in claim 14 wherein the laser beam is a pulsed femtosecond laser beam.

16. A method as recited in claim 14 wherein the microchannel extends through the trabecular meshwork and is positioned to interconnect the anterior chamber of the eye in fluid communication with Schlemm's canal.

17. A method as recited in claim 16 wherein the microchannel is positioned to enhance the functionality of Schlemm's canal by improving the outflow of aqueous fluid from the anterior chamber of the eye in order to lower the intraocular pressure (IOP) of the eye.

18. A method as recited in claim 14 wherein the creating step is accomplished by an Optical Coherence Tomography (OCT) device.

19. A method as recited in claim 14 wherein the performance of the method is controlled by a computer.

20. A method as recited in claim 14 wherein the laser beam path of the directing step passes through the sclera.

21. A computer program product comprising program sections for respectively: defining a microchannel in a transparent material; creating an image of the microchannel in the material; directing a laser beam along a laser beam path to a focal point in the material; guiding the focal point to alter material by Laser Induced Optical Breakdown (LIOB) to create the microchannel; determining a deviation of the focal point from the image of the microchannel; producing an error signal indicative of the deviation; and moving the focal point of the laser beam to minimize the error signal during creation of the microchannel.
